SEQUENCE LISTING (31 sequences)

SEQ ID NO: 1 - DNA, 1416 bp, cDNA Sequence
  misc_feature (19)..(19): n=a
gcccactcgctgcggcctntctggctccagaccgccctccggatcggaccctgcgaatggttttggctatatcttcatgtaggacctactccctatcccgtcggccgcggctgggcttcatcaccaggcctcctcacagattcctgtcccttctgtgtcctggactccggatacctcaactctcagtactttgtgctcagcccaggcccagagccatggctatctcctcttcctcctgcgaactgcccctggtggctgtgtgccaggtaacatcgacgccagacaagcaacagaactttaaaacatgtgctgagctggttcgagaggctgccagactgggtgcctgcctggctttcctgcctgaggcatttgacttcattgcacgggaccctgcagagacgctacacctgtctgaaccactgggtgggaaacttttggaagaatacacccagcttgccagggaatgtggactctggctgtccttgggtggtttccatgagcgtggccaagactgggagcagactcagaaaatctacaattgtcacgtgctgctgaacagcaaaggggcagtagtggccacttacaggaagacacatctgtgtgacgtagagattccagggcaggggcctatgtgtgaaagcaactctaccatgcctgggcccagtcttgagtcacctgtcagcacaccagcaggcaagattggtctagctgtctgctatgacatgcggttccctgaactctctctggcattggctcaagctggagcagagatacttacctatccttcagcttttggatccattacaggcccagcccactgggaggtgttgctgcgggcccgtgctatcgaaacccagtgctatgtagtggcagcagcacagtgtggacgccaccatgagaagagagcaagttatggccacagcatggtggtagacccctggggaacagtggtggcccgctgctctgaggggccaggcctctgccttgcccgaatagacctcaactatctgcgacagttgcgccgacacctgcctgtgttccagcaccgcaggcctgacctctatggcaatctgggtcacccactgtcttaagacttgacttctgtgagtttagacctgcccctcccacccccaccctgccactatgagctagtgctcatgtgacttggaggcaggatccaggcacagctcccctcacttggagaaccttgactctcttgatggaacacagatgggctgcttgggaaagaaactttcacctgagcttcacctgaggtcagactgcagtttcagaaaggtggaattttatatagtcattgtttatttcatggaaactgaagttctgctgagggctgagcagcactggcattgaaaaatataataatcataaagtcaaaaaaaaaaaaaaaaaaaaaaa

SEQ ID NO: 2 - DNA, 23 bp, Homo sapiens
tctgaaactgcagtctgacctca

SEQ ID NO: 3 - DNA, 21 bp, Homo sapiens
caggcacagctcccctcactt

SEQ ID NO: 4 - DNA, 20 bp, Homo sapiens
  misc_feature (3)..(3): n is a, c, g, or t
gtngtnccnggncaygtngt

SEQ ID NO: 5 - DNA, 26 bp, Homo sapiens
  misc_feature (6)..(6): n is a, c, g, or t
acrtgnacrtgyttnacngtytgngc

SEQ ID NO: 6 - DNA, 21 bp, Drosophila melanogaster
gcgcctttgtggcctcgactg

SEQ ID NO: 7 - DNA, 21 bp, Drosophila melanogaster
cggtggcggaagttgtctggt

SEQ ID NO: 8 - DNA, 20 bp, Caenorhabditis elegans
gtggcggctgctcaaactgg

SEQ ID NO: 9 - DNA, 21 bp, Caenorhabditis elegans
tcgcgacgatgaacaagtcgg

SEQ ID NO: 10 - DNA, 19 bp, Homo sapiens
gccctccggatcggaccct

SEQ ID NO: 11 - DNA, 20 bp, Homo sapiens
gacctactccctatcccgtc

SEQ ID NO: 12 - DNA, 21 bp, Homo sapiens
gctgcgaagtgcacagctaag

SEQ ID NO: 13 - DNA, 24 bp, Homo sapiens
aaactgaagcctctttcctctgac

SEQ ID NO: 14 - DNA, 20 bp, Homo sapiens
tgggcttcatcaccaggcct

SEQ ID NO: 15 - DNA, 22 bp, Homo sapiens
ctgggctgagcacaaagtactg

SEQ ID NO: 16 - DNA, 21 bp, Homo sapiens
gcttgtctggcgtcgatgtta

SEQ ID NO: 17 - DNA, 36 bp, Homo sapiens
tgacgtcgacatatgtcaactctagttaataccacg

SEQ ID NO: 18 - DNA, 25 bp, Homo sapiens
tgggtacctcgactagcttatgtcc

SEQ ID NO: 19 - protein, 147 aa, Homo sapiens
  misc_feature (82)..(82): Xaa is an unknown amino acid
MSFRFGQHLIKPSVVFLKTELSFALVNRKPVVPGHVLVCPLRPVERFHDLRPDEVADLFQTTQRVGTVVEKHFHGTSLTFSXQDGPEAGQTVKHVHVHVLPRKAGDFHRNDSIYEELQKHDKEDFPASWRSEEEEAAEAAALRVYFQ

SEQ ID NO: 20 - protein, 150 aa, murine
MSFRFGQHLIKPSVVFLKTELSFALVNRKPVVPGHVLVCPLRPVERFRDLHPDEVADLFQVTQRVGTVVEKHFQGTSITFSMQDGPEAGQTVKHVHVHVLPRKAGDFPRNDNIYDELQKHDREEEDSPAFWRSEKEMAAEAEALRVYFQA

SEQ ID NO: 21 - protein, 327 aa, Homo sapiens
MLGFITRPPHRFLSLLCPGLRIPQLSVLCAQPRPRAMAISSSSCELPLVAVCQVTSTPDKQQNFKTCAELVREAARLGACLAFLPEAFDFIARDPAETLHLSEPLGGKLLEEYTQLARECGLWLSLGGFHERGQDWEQTQKIYNCHVLLNSKGAVVATYRKTHLCDVEIPGQGPMCESNSTMPGPSLESPVSTPAGKIGLAVCYDMRFPELSLALAQAGAEILTYPSAFGSITGPAHWEVLLRARAIETQCYVVAAAQCGRHHEKRASYGHSMVVDPWGTVVARCSEGPGLCLARIDLNYLRQLRRHLPVFQHRRPDLYGNLGHPLS

SEQ ID NO: 22 - protein, 323 aa, murine
MLGFITRPPHQLLCTGYRLLRIPVLCTQPRPRTMSSSTSWELPLVAVCQVTSTPNKQENFKTCAELVQEAARLGACLAFLPEAFDFIARNPAETLLLSEPLNGDLLGQYSQLARECGIWLSLGGFHERGQDWEQNQKIYNCHVLLNSKGSVVASYRKTHLCDVEIPGQGPMRESNYTKPGGTLEPPVKTPAGKVGLAICYDMRFPELSLKLAQAGAEILTYPSAFGSVTGPAHWEVLLRARAIESQCYVIAAAQCGRHHETRASYGHSMVVDPWGTVVARCSEGPGLCLARIDLHFLQQMRQHLPVFQHRRPDLYGSLGHPLS

SEQ ID NO: 23 - protein, 460 aa, Drosophila melanogaster
MSTLVNTTRRSIVIAIHQQLRRMSVQKRKDQSATIAVGQMRSTSDKAANLSQVIELVDRAKSQNACMLFLPECCDFVGESRTQTIELSEGLDGELMAQYRELAKCNKIWISLGGVHERNDQKIFNAHVLLNEKGELAAVYRKLHMFDVTTKEVRLRESDTVTPGYCLERPVSTPVGQIGLQICYDLRFAEPAVLLRKLGANLLTYPSAFTYATGKAHWEILLRARAIETQCFVVAAAQIGWHNQKRQSWGHSMIVSPWGNVLADCSEQELDIGTAEVDLSVLQSLYQTMPCFEHRRNDIYALTAYNLRSKEPTQDRPFATNIVDKRTIFYESEHCFAFTNLRCVVKGHVLVSTKRVTPRLCGLDCAEMADMFTTVCLVQRLLEKIYQTTSATVTVQDGAQAGQTVPHVHFHIMPRRLGDFGHNDQIYVKLDERAEEKPPRTIEERIEEAQIYRKFLTDIS

SEQ ID NO: 24 - protein, 440 aa, C. elegans
MLSTVFRRTMATGRHFIAVCQMTSDNDLEKNFQAAKNMIERAGEKKCEMVFLPECFDFIGLNKNEQIDLAMATDCEYMEKYRELARKHNIWLSLGGLHHKDPSDAAHPWNTHLIIDSDGVTRAEYNKLHLFDLEIPGKVRLMESEFSKAGTEMIPPVDTPIGRLGLSICYDVRFPELSLWNRKRGAQLLSFPSAFTLNTGLAHWETLLRARAIENQCYVVAAAQTGAHNPKRQSYGHSMVVDPWGAVVAQCSERVDMCFAEIDLSYVDTLREMQPVFSHRRSDLYTLHINEKSSETGGLKFARFNIPADHIFYSTPHSFVFVNLKPVTDGHVLVSPKRVVPRLTDLTDAETADLFIVAKKVQAMLEKHHNVTSTTICVQDGKDAGQTVPHVHIHILPRRAGDFGDNEIYQKLASHDKEPERKPRSNEQMAEEAVVYRNLM

SEQ ID NO: 25 - protein, 362 aa, Polypeptide Sequence
  misc_feature (6)..(6): Xaa is an unknown amino acid
PLAAAXLAPDRPPDRTLRMVLAISSCRTYSLSRRPRLGFITRPPHRFLSLLCPGLRIPQLSVLCAQPRPRAMAISSSSCELPLVAVCQVTSTPDKQQNFKTCAELVREAARLGACLAFLPEAFDFIARDPAETLHLSEPLGGKLLEEYTQLARECGLWLSLGGFHERGQDWEQTQKIYNCHVLLNSKGAVVATYRKTHLCDVEIPGQGPMCESNSTMPGPSLESPVSTPAGKIGLAVCYDMRFPELSLALAQAGAEILTYPSAFGSITGPAHWEVLLRARAIETQCYVVAAAQCGRHHEKRASYGHSMVVDPWGTVVARCSEGPGLCLARIDLNYLRQLRRHLPVFQHRRPDLYGNLGHPLS

SEQ ID NO: 26 - protein, 23 aa, Homo sapiens
DLTSVSLDLPLPPPPCHYELVLM

SEQ ID NO: 27 - protein, 15 aa, Homo sapiens
LGGRIQAQLPSLGEP

SEQ ID NO: 28 - protein, 13 aa, Homo sapiens
WNTDGLLGKETFT

SEQ ID NO: 29 - protein, 24 aa, Homo sapiens
ASPEVRLQFQKGGILYSHCLFHGN

SEQ ID NO: 30 - protein, 5 aa, Homo sapiens
SSAEG

SEQ ID NO: 31 - protein, 20 aa, Homo sapiens
AALALKNIIIIKSKKKKKKK

(19) United States
(12) Patent Application Publication
(10) Pub. No.: US 2005/0019890 A1
(43) Pub. Date: Jan. 27, 2005

(54) Nitrilase homologs

(75) Inventor: Carlo M. Croce, Philadelphia, PA (US)

Correspondence Address: DRINKER BIDDLE & REATH, ONE LOGAN SQUARE, 18TH AND CHERRY STREETS, PHILADELPHIA, PA, 19103-6996, US

(73) Assignee: Thomas Jefferson University, Philadelphia, PA (US)

(21) Appl. No.: 10/923,960

(22) Filed: Aug. 23, 2004

Related U.S. Application Data
(63) Continuation of application No. 09/357,675, filed on Jul. 20, 1999, now abandoned.
(60) Provisional application No. 60/093,350, filed on Jul. 20, 1998.

(51) Int. Cl. C12N 9/80; C12Q 1/68; C07H 21/04

(52) U.S. Cl. 435/228; 435/6; 435/69.1; 435/320.1; 435/325; 536/23.2

(57) ABSTRACT

The present invention relates to nucleotide sequences of the NIT1 gene and amino acid sequences of its encoded proteins, as well as derivatives and analogs thereof. Additionally, the present invention relates to the use of nucleotide sequences of NIT1 genes and amino acid sequences of their encoded proteins, as well as derivatives and analogs thereof and antibodies thereto, as diagnostic and therapeutic reagents for the detection and treatment of cancer. The present invention also relates to therapeutic compositions comprising Nit1 proteins, derivatives or analogs thereof, antibodies thereto, nucleic acids encoding the Nit1 proteins derivatives, or analogs and NIT1 antisense nucleic acids, and vectors containing the NIT1 coding sequence.

### FIELD OF THE INVENTION

The present invention generally relates to the field of oncology and tumor suppressor genes, and more particularly to the structure and function of the NIT1 gene, the structure of its encoded proteins, and the use of NIT1 genes and the NIT1 related genes and their encoded proteins and vectors containing the NIT1 coding sequence as diagnostic and therapeutic reagents for the detection and treatment of cancer.

### BACKGROUND OF THE INVENTION

Introduction

The present invention relates to nucleotide sequences of the NIT1 gene and amino acid sequences of its encoded proteins, as well as derivatives and analogs thereof. Additionally, the present invention relates to the use of nucleotide sequences of NIT1 genes and amino acid sequences of their encoded proteins and vectors containing the NIT1 coding sequence, as well as derivatives and analogs thereof and antibodies thereto, as diagnostic and therapeutic reagents for the detection and treatment of cancer. The present invention also relates to therapeutic compositions comprising NIT1 proteins, derivatives or analogs thereof, antibodies thereto, nucleic acids encoding the Nit1 proteins, derivatives, or analogs, and NIT1 antisense nucleic acids, and vectors containing the NIT1 coding sequence.

Approaches to Elucidation and Characterization of NIT1

The tumor suppressor gene FHIT encompasses the common human chromosomal fragile site at 3p14.2 and numerous cancer cell bi-allelic deletions. To study Fhit function, Fhit genes in D. melanogaster and C. elegans were cloned and characterized. The Fhit genes in both of these organisms code for fusion proteins in which the Fhit domain is fused with a novel domain showing homology to bacterial and plant nitrilases; the D. melanogaster fusion protein exhibited diadenosine triphosphate (ApppA) hydrolase activity expected of an authentic Fhit homolog.

In human and mouse, the nitrilase homologs and Fhit are encoded by two different genes, FHIT and NIT1, localized on chromosomes 3 and 1 in human, and 14 and 1 in mouse, respectively. Human and murine NIT1 genes were cloned and characterized, their exon-intron structure, their patterns of expression, and their alternative mRNA processing were determined.

The tissue specificity of expression of murine FHIT and NIT1 genes was nearly identical. Typically, fusion proteins with dual or triple enzymatic activities have been found to carry out specific steps in a given biochemical or biosynthetic pathway; Fhit and Nit1, as fusion proteins with dual or triple enzymatic activities, likewise collaborate in a biochemical or cellular pathway in mammalian cells.

Importance of FHIT

The human FHIT gene at chromosome 3p14.2, spanning the constitutive chromosomal fragile site FRA3B, is often altered in the most common forms of human cancer and is a tumor suppressor gene. The human FHIT gene is greater than one megabase in size encoding an mRNA of 1.1 kilobases and a protein of 147 amino acids.

The rearrangements most commonly seen are deletions within the gene. These deletions, often occurring independently in both alleles and resulting in inactivation, have been reported in tumor-derived cell lines and primary tumors of lung, head and neck, stomach, colon, and other organs. In cell lines derived from several tumor types, DNA rearrangements in the FHIT locus correlated with RNA and/or Fhit protein alterations.

Because the inactivation of the FHIT gene by point mutations has not been demonstrated conclusively and because several reports have shown the amplification of aberrant-sized FHIT reverse transcription-PCR (RT-PCR) products from normal cell RNA, a number of investigators have suggested that the FHIT gene may not be a tumor suppressor gene. On the other hand it has been reported that re-expression of Fhit in lung, stomach and kidney tumor cell lines lacking endogenous protein suppressed tumorigenicity in vivo in 4 out of 4 cancer cell lines. This suggests that FHIT is indeed a tumor suppressor gene. It is noted that a report has suggested that Fhit enzymatic activity is not required for its tumor suppressor function.

Fhit protein is a member of the histidine triad (HIT) superfamily of nucleotide binding proteins and is similar to the Schizosaccharomyces pombe diadenosine tetraphosphate (Ap₄A) hydrolase. Additionally it has been reported that, in vitro, Fhit has diadenosine triphosphate (ApppA) hydrolase enzymatic activity.

Neither the in vivo function of Fhit nor the mechanism of its tumor suppressor activity is known. Nonetheless, genetic, biochemical and crystallographic analysis suggest that the enzyme-substrate complex is the active form that signals for tumor suppression. One approach to investigate function is to investigate Fhit in model organisms such as Drosophila melanogaster and Caenorhabditis elegans.

The present invention involves the isolation and characterization of the NIT1 gene in these organisms. Fhit occurs in a fusion protein, Nit-Fhit, in D. melanogaster and C. elegans, but FHIT and NIT1 are separate genes in mammalian cells. The human and mouse NIT1 genes are members of an uncharacterized mammalian gene family with homology to bacterial and plant nitrilases, enzymes which cleave nitriles and organic amides to the corresponding carboxylic acids plus ammonia.

### SUMMARY OF THE INVENTION

Accordingly, it is an object of the present invention to purify a NIT1 gene.

It is a further object of the present invention to purify a NIT1 gene, wherein the purified gene is a human gene.

It is an object of the present invention to purify a NIT1 gene, wherein the purified gene is a mammalian gene.

It is an object of the present invention to purify a Nit1 protein.

It is another object of the present invention to purify a Nit1 protein, wherein the purified protein is a human protein.

It is another object of the present invention to purify a Nit1 protein, wherein the purified protein is a mammalian protein.

Yet another aspect of the present invention is a purified protein encoded by a nucleic acid having a nucleotide sequence consisting of the coding region of SEQ ID NO:1 (FIG. 6).

Another aspect of the present invention is an antibody capable of binding a Nit1 protein.

It is another object of the present invention to isolate a nucleic acid of less than 100 kb, comprising a nucleotide sequence encoding a Nit 1 protein.

Another object of the present invention is a pharmaceutical composition comprising a therapeutically effective amount of a Nit1 protein; and a therapeutically acceptable carrier.

Another object of the present invention is a method of treating or preventing a disease or disorder in a subject comprising administering to said subject a therapeutically effective amount of a molecule that inhibits Nit1 function.

Another aspect of the present invention is a method of treating or preventing a disease or disorder in a subject comprising administering to said subject a therapeutically effective amount of a molecule that enhances Nit1 function.

It is yet another aspect of the present invention to diagnose or screen for the presence of or a disposition for developing a disease in a subject, comprising detecting one or more mutations in NIT1 DNA, RNA or Nit1 protein derived from the subject in which the presence of said one or more mutations indicates the presence of the disease or disorder or a predisposition for developing the disease or disorder.

It is yet another aspect of the present invention to treat a disease or disorder with a vector containing the coding segment of the NIT1 gene.

### BRIEF DESCRIPTION OF THE DRAWINGS

FIG. 1. A sequence comparison of human (Fhit SEQ ID NO:19 and Nit1 SEQ ID NO:21), murine (Fhit SEQ ID NO:20 and Nit1 SEQ ID NO:22), D. melanogaster (NitFhit SEQ ID NO:23), and C. elegans (NitFhit SEQ ID NO:23) Nit1 and Fhit proteins. Identities are shown in black boxes, similarities are shown in shaded boxes. For human and mouse FHIT, GenBank accession numbers are U46922 and AF047699, respectively.

FIG. 2. Northern blot analysis of expression of NIT1 and FHIT mRNAs in murine and human tissues, as well as in D. melanogaster, and C. elegans. (A) Mouse multiple tissues Northern blot. Lanes 1-8: heart, brain, spleen, lung, liver, skeletal muscle, kidney, and testis. (Top) Fhit probe; (Middle) Nit1 probe; (Bottom) actin probe. (B) Human blot, NIT1 probe. Lanes 1-8: heart, brain, placenta, lung, liver, skeletal muscle, kidney, and pancreas. (C) Lanes 1 and 2: D. melanogaster adult, D. melanogaster embryo; D. melanogaster Nit-Fhit probe. Lane 3: C. elegans adult; C. elegans Nit-Fhit probe.

FIG. 3. Genomic organization of human and murine NIT1 genes and D. melanogaster and C. elegans Nit-Fhit genes. (A) Exon-intron structure of the genes. (B) Alternative processing of human NIT1 gene.

FIG. 4. Cleavage of ApppA by D. melanogaster Nit-Fhit. At indicated times of incubation, samples were spotted on TLC plates with appropriate nucleotide standards.

FIG. 5. Analysis of alternative transcripts of human NIT1 by RT-PCR. RT-PCR of HeLa RNA was performed with primers in different exons. Lanes 1-6: exons 1 and 3 (transcript 2); exons 1C and 3 (transcript 5); exons 1A and 3 (transcripts 3, upper band and 4, lower band): exons 2 and 3 (transcripts 2-4); exons 1 and 1C (transcript 5); and exons 1 and 2 (transcript 2).

FIG. 6. A nucleotide sequence (SEQ ID NO: 1) and the polypeptides and peptides deduced from the nucleotide sequence (SEQ ID NO:25 through SEQ ID NO:31).

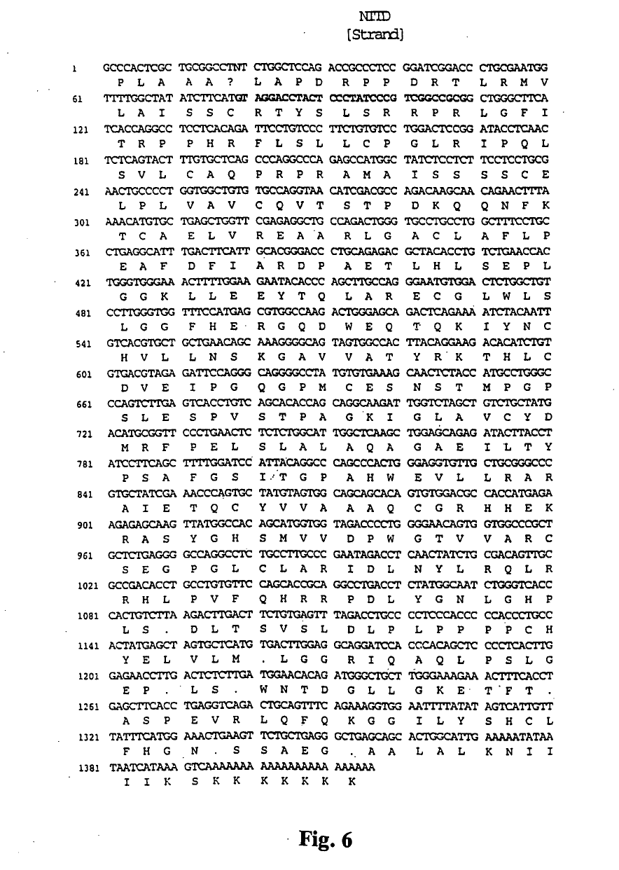

### DETAILED DESCRIPTION

Genomic and cDNA Clones

One million plaques of a mouse genomic library (bacteriophage library from strain SVJ129, Stratagene, La Jolla, Calif.) and one hundred thousand plaques of a D. melanogaster genomic library were screened with corresponding cDNA probes. Clones were purified and DNA was isolated. Sequencing was carried out using Perkin Elmer thermal cyclers and ABI 377 automated DNA sequencers. DNA pools from a human BAC library (Research Genetics, Huntsville, Ala.) were screened by PCR with NIT1 primers (TCTGAAACTGCAGTCTGACCTCA (SEQ ID NO:2) and CAGGCACAGCTCCCCTCACTT (SEQ ID NO:3)) according to the supplier's protocol. The DNA from the positive clone, 31K11, has been isolated using standard procedures and sequenced. Chromosomal localization of the human NIT1 gene was determined using a radiation hybrid mapping panel (Research Genetics) according to the supplier's protocol and with the same primers as above. To map marine Nit1 gene, Southern blot analysis of genomic DNA from progeny of a (AEJ/Gn-a bp^{H}/a bp^{H} x M, spretus) F1 x AEJ/Gn-a bp^{h}/a bp^{h} backcross was performed using a full length murine Nit1 cDNA probe. This probe detected a unique 2.0 kb DraI fragment in AEJ DNA and a unique 0.75 kb fragment in M. spretus DNA. Segregation of these fragments were followed in 180 N2 offspring of the backcross. Additional Mit markers (D1Mit34, D1Mit35, and D1Mit209) were typed from DNA of 92 mice by using PCR consisting of an initial denaturation of 4 minutes at 94° C. followed by 40 cycles of 94° C. for 30 seconds, 55° C. for 30 seconds and 72° C. for 30 seconds. Linkage analysis was performed using the computer program SPRETUS MADNESS: PART DEUX. Human and mouseNIT1 expressed sequence tag (EST) clones were purchased form Research Genetics. The sequences of human and murine NIT1 genes and cDNAs and D. melanogaster and C. elegans Nit-Fhit cDNAs have been deposited in GenBank.

In Situ Hybridization

D. melanogaster polytene chromosome spreads were prepared from salivary glands of third-instar larvae as described. NitFhit DNA fragments were labeled with digoxigenin-11-dUTP using a random-primed DNA labeling kit (Boeringer Mannheim, Indianapolis, Ind.), and were used as probes for the chromosomal in situ hybridization. Hybridization was for 20 hours at 37° C. in hybridization buffer: 50% formamide, 2× standard saline citrate (SSC), 10% dextran sulfate, 400 mg/ml salmon sperm DNA. Antidigoxigenin-fluorescein antibodies (Boehringer Mannheim) were used for detection of hybridizing regions. DNA was counterstained with Hoechst 33258 (Sigma, St. Louis, Mo.). The slides were analyzed by fluorescence microscopy. For in situ hybridization, embryos were fixed and processed as described previously, except that single-stranded RNA probes were used. Full length NitFhit cDNA was cloned into BluescriptII KS+ vector and used to synthesize antisense RNA probes with the Genius 4 kit (Boehringer Mannheim).

RT-PCR, Northern and RACE Analysis

Human and mouse multiple tissue northern blots (Clontech, Palo Alto, Calif.) were hybridized with corresponding NIT1 cDNA probes and washed using the supplier's protocol. For the HeLa cell line, total RNA was isolated from 1-5×10⁸ cells using Trizol reagent (Gibco BRL, Gaithersburg, Md.). D. melanogaster PolyA+ RNA was purchased from Clontech. Three μg of polyA+ RNA or 15 μg of total RNA were electrophoresed in 0.8% agarose in a borate buffer containing formaldehyde, transferred to HybondN+ membrane (Amersham, Arlington Heights, Ill.) using standard procedures and hybridized as described above. For RT-PCR, 200 ng of polyA+ RNA or 3 μg of total RNA were treated with DNaseI (amplification grade, Gibco BRL) following the manufacturer's protocol. DNase-treated RNA was used in reverse transcription (RT) reactions as follows: 10 nM each dNTP, 100 pmoles random hexamers (oligo (dT) priming was used in some cases), DNaseI treated RNA, and 200 units of murine leukemia virus (MuLV) reverse transcriptase (Gibco BRL), in total volume of 20 μl were incubated at 42° C. for 1 hour followed by the addition of 10 μg RNase A and incubation at 37° C. for 30 min. One μl of the reaction was used for each PCR reaction. PCR reactions were carried out under standard conditions using 10 pmoles of each gene-specific primer and 25-35 cycles of 95° 30″, 55-60° 30″, 72° 1′. Products were separated on 1.5% agarose gels and sometimes isolated and sequenced or cloned and sequenced. Oligo (dT)-primed double-stranded cDNA was synthesized by using procedures and reagents from the Marathon RACE cDNA amplification kit (Clontech); the cDNA was ligated to Marathon adapters (Clontech). 3′ and 5′ RACE products were generated by long PCR using gene-specific primers and the AP1 primer (Clontech). To increase the specificity of the procedure, the second PCR reaction was carried out by using nested gene-specific primers and the AP2 primer (Clontech). PCR reactions were performed according to the Marathon protocol using the Expand long template PCR system (Boehringer Mannheim) and 30 cycles of: 94° 30″, 60° 30″, 68° 4′. RACE products were electrophoresed, identified by hybridization and sequenced. Degenerate FHIT primers were: GTNGTNCCNGGNCAYGTNGT (SEQ ID NO:4) and ACRTGNACRTGYTTNACNGTYTGNGC (SEQ ID NO:5). D. Melanogaster Fhit RACE and RT-PCR primers were: GCGCCTTTGTGGCCTCGACTG (SEQ ID NO:6) and CGGTGGCGGAAGTTGTCTGGT (SEQ ID NO:7). C. elegans Fhit RACE and RT-PCR primers were: GTGGCGGCTGCTCAAACTGG (SEQ ID NO:8) and TCGCGACGATGAACAAGTCGG (SEQ ID NO:9). Human NIT1 RT-PCR primers were: GCCCTCCGGATCGGACCCT (SEQ ID NO:10) (exon 1); GACCTACTCCCTATCCCGTC (SEQ ID NO:11) (exon 1a); GCTGCGAAGTGCACAGCTAAG (SEQ ID NO:12) and AAACTGAAGCCTCTTTCCTCTGAC (SEQ ID NO:13) (exon 1c); TGGGCTTCATCACCAGGCCT (SEQ ID NO:14) and CTGGGCTGAGCACAAAGTACTG (SEQ ID NO:15) (exon 2); GCTTGTCTGGCGTCGATGTTA (SEQ ID NO:16) (exon 3).

Protein Expression and Enzymatic Characterization

The NIT-FHIT cDNA was amplified with primers TGACGTCGACATATGTCAACTCTAGTTAATACCACG (SEQ ID NO:17) and TGGGTACCTCGACTAGCTTATGTCC (SEQ ID NO:18), digested with NdeI and KpnI, and cloned into plasmid pSGA02 as a Nde1-Kpn1 fragment. Escherichia coli strain SG100 transformants were grown in Luria-Bertani with 100 μg/ml of ampicillin and 15 μg/ml of chloramphenicol at 15° C. When the culture reached an optical density (600 nm) of 0.25, isopropyl β-D-thiogalactoside was added to a final concentration of 200 μM. NitFhit protein was purified from inclusion bodies as described. Briefly, the cell pellet from a 1-liter culture was resuspended in 50 ml of 20 mM Tris.HCl (pH 7.5), 20% sucrose, 1 mM EDTA and repelleted. Outer cell walls were lysed by resuspension in ice-water. Spheroblasts were pelleted, resuspended in 140 mM NaCl, 2.7 mM KCl, 12 mM Na.P04 (pH 7.3), 5 mM EDTA, 500 mM phenylmethylsulfonyl fluoride, 1 μg/ml leupeptin and 20 pmg/ml of aprotinin, and sonicated. The resulting inclusion body preparation was washed and solubilized in 5 M guanidinium hydrochloride, 50 mM Tris.HCl (pH 8.0), 5 mM EDTA. Soluble NitFhit protein was added dropwise to 250 ml of 50 mM Tris.HCl (pH 8.0), 1 mM DTT, 20% glycerol at 40° C. After a 14 hour incubation, the 13-kg supernatant was concentrated 100-fold with a Centricon filter. A 1-liter culture yielded approximately 200 μg of partially purified, soluble NitFhit. ApppA hydrolase activity was assayed at 30° C. in 20 μl of 50 mM Na.HEPES pH 7.5, 10% glycerol, 0.5 mM MnCI2, 4 mM ApppA, 1 μM NitFhit. TLC plates were developed as described.

Cloning and Characterization of D. melanogaster and C. elegans Fhit Homologs

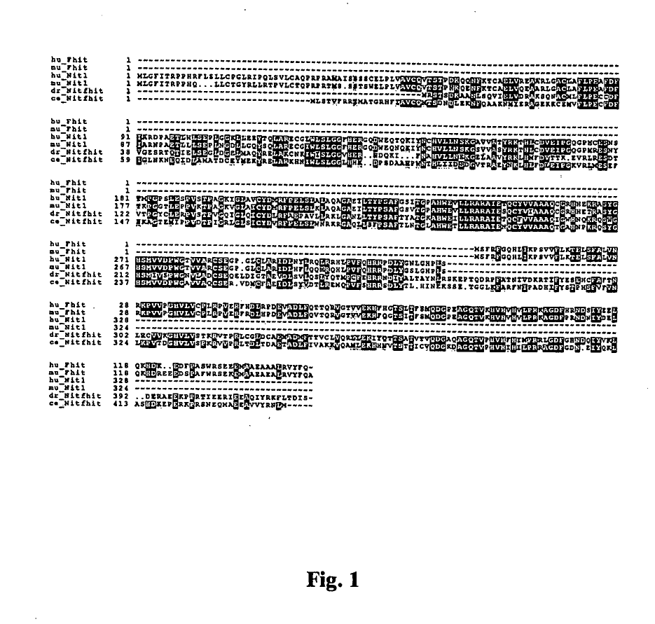

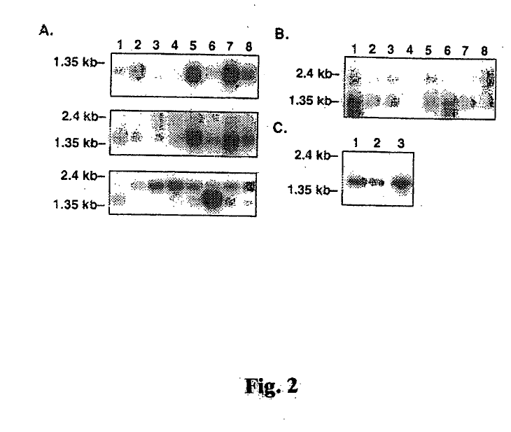

To obtain D. melanogaster Fhit sequences, degenerate primers were designed in the conserved regions of exons 5 and 7 of human FHIT. RT-PCR experiments with these primers and D. melanogaster RNA resulted in an ˜200 bp product, which when translated showed ˜50% identity to human Fhit protein. This sequence was used to design specific D. melanogaster Fhit primers. 5′ and 3′ RACE with these primers resulted in ˜1.5 kb full length cDNA (including polyadenylation signal and Poly(A) tail) encoding a 460 amino acid protein with a 145 amino acid C-terminal part homologous to human Fhit (40% identity and 47% similarity) and a 315 amino acid N-terminal extension (FIG. 1). Northern analysis (FIG. 2C) showed a singer band of ˜1.5 kb in both embryo and adult D. melanogaster confirming that the full length cDNA has been cloned.

The 460 amino acid predicted protein sequence was used in a BLASTP search. Of the top 50 scoring alignments, 22 aligned with the 145 residue C-terminal segment (Fhit-related sequences) and 28 aligned with the 315 residue N-terminal segment. The 28 sequences aligning with the N-terminus were led by an uncharacterized gene from chromosome X of Saccharomyces cerevisiae (P-value of 1.4×10⁻⁴⁵), followed by uncharacterized ORFs of many bacterial genomes and a series of enzymes from plants and bacteria that have been characterized as nitrilases and amidases. Thus, the 460 amino acid predicted protein contains an N-terminal nitrilase domain and a C-terminal Fhit domain and was designated NitFhit.

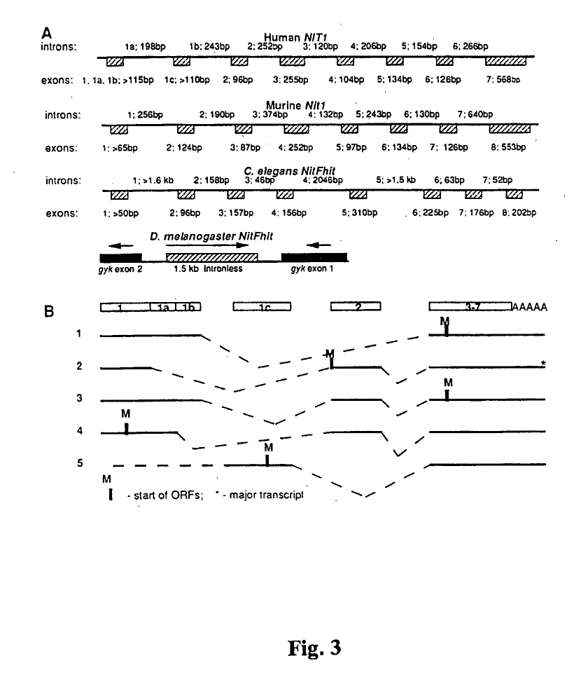

The D. melanogaster Nit-Fhit cDNA probe was used to screen a D. melanogaster lambda genomic library. Sequencing of positive clones revealed that the gene is intronless and, interestingly, the 1.5-kb Nit-Fhit gene is localized within the 1.6-kb intron 1 of the D. melanogaster homolog of the murine glycerol kinase (Gyk) gene. The direction of transcription of the Nit-Fhit gene is opposite to that of the Gyk gene (FIG. 3A). It is not known if such localization affects transcriptional regulation of these two genes.

The cytological position of the Nit-Fhit gene was determined by in situ hybridization to salivary gland polytene chromosomes. These experiments showed that there is only one copy of the sequence which was localized to region 61A, at the tip of the left arm of chromosome 3. Digoxigenin-labeled RNA probes were hybridized to whole-mount embryos to determine the pattern of expression during development. Nit-Fhit RNA was uniformly expressed throughout the embryo suggesting that NitFhit protein could be important for most of the embryonic cells.

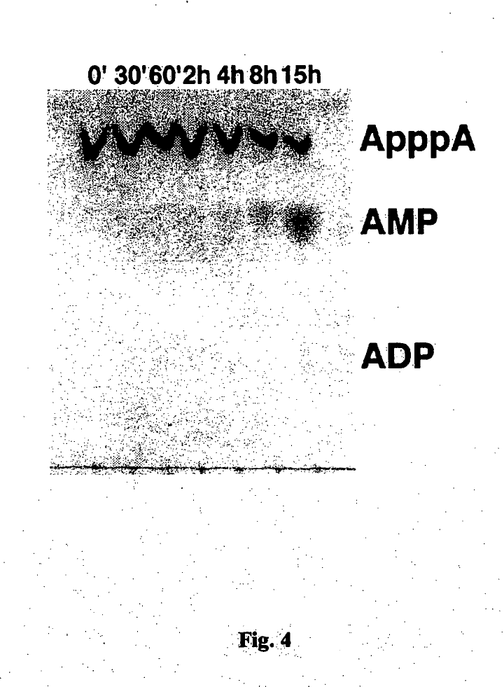

Because human Fhit protein and the D. melanogaster Fhit domain were only 40% identical, to show that the authentic D. melanogaster Fhit homolog was cloned, its enzymatic activity was tested. FIG. 4 shows that recombinant D. melanogaster NitFhit is capable of cleaving ApppA to AMP and ADP and therefore possesses ApppA hydrolase activity.

C. elegans

Fhit genomic sequences were obtained from the Sanger database (contig Y56A3) by using BLAST searches. 5′ and 3′ RACE with C. elegans Fhit specific primers yielded a 1.4-kb cDNA (including polyadenylation signal and Poly(A) tail) coding for a 440 amino acid protein (FIG. 1). Northern analysis (FIG. 2C) showed a single band of a similar size in adult worms. Similarly to D. melanogaster, the C. elegans protein contained an N-terminal nitrilase domain and a C-terminal Fhit domain (FIG. 1) with 50% identity and 57% similarity to human Fhit. Comparison between C. elegans Nit-Fhit cDNA and genomic sequences from the Sanger database revealed that the C. elegans Nit-Fhit gene comprises 8 exons and is more than 6.5 kb in size (FIG. 3A); the nitrilase domain is encoded by exons 1-6, and the Fhit domain is encoded by exons 6-8. D. melanogaster and C. elegans NitFhit proteins are 50% identical and 59% similar and exhibit several conserved domains (FIG. 1).

Cloning and Characterized of Human and Murine NIT cDNAs and Genes

Because Fhit and nitrilase domains are part of the same polypeptides in D. melanogaster and C. elegans, it is reasonable to suggest that they may be involved in the same biochemical or cellular pathway(s) in these organisms. Because nitrilase homologs are conserved in animals, the mammalian nitrilase homologs were cloned as candidate Fhit-interacting proteins.

To obtain human and murine NIT1 sequences, the D. melanogaster nitrilase domain sequence was used in BLAST searches of the GenBank EST database. Numerous partially sequenced human and murine NIT1 ESTs were found. All mouse NIT1 ESTs were identical, as were all human NIT1 ESTs, suggesting the presence of a single Nit1 gene in mouse and human. To obtain the full-length human and mouse cDNAs, several human and mouse ESTs and human 5′ and 3′ RACE products were completely sequenced. This resulted in the isolation of a 1.4-kb full-length human sequence encoding 327 amino acids and a ˜1.4-kb mouse full-length sequence coding for 323 amino acids (FIG. 1), although several alternatively spliced products were detected in both cases (see below and FIG. 3B). Both cDNAs are polyadenylated, but lack polyadenylation signals, although AT-rich regions are present at the very 3′ end of each cDNA. Mouse and human Nit1 amino acid sequences were 90% identical; the human Nit1 amino acid sequence was 58% similar and 50% identical to the C. elegans nitrilase domain and 63% similar and 53% identical to the D. melanogaster nitrilase domain (FIG. 1).

Murine lambda and human BAC genomic libraries were screened with the corresponding NIT1 cDNA probes, yielding one mouse lambda clone and one human BAC clone containing the NIT1 genes. The human and murine NIT1 genomic regions were sequenced and compared to the corresponding cDNA sequences. The genomic structure of human and mouse NIT1 genes is shown in FIG. 3A. Both genes are small: the human gene is ˜3.2 kb in size and contains 7 exons; the murine gene is 3.6 kb in size and contains 8 exons. Southern analysis confirmed that both human and mouse genomes harbor a single NIT1 gene.

A radiation hybrid mapping panel (GeneBridge 4) was used to determine the chromosomal localization of the human NIT1 gene. By analysis of PCR data at the Whitehead/MIT database on the world wide web at genome.wi.mit.edu, the NIT1 gene was localized 6.94 cR from the marker CHLC.GATA43A4, which is located at 1q21-1q22.

A full length murine Nit1 cDNA probe was used to determine the chromosomal location of the murine gene by linkage analysis. Interspecific backcross analysis of 180 N₂ mice demonstrated that the Nit1 locus cosegregated with several previously mapped loci on distal mouse chromosome 1. The region to which Nit1 maps was further defined by PCR of genomic DNA from 92 N₂ mice using the markers D1Mit34, D1Mit35 and D1Mit209 (Research Genetics). The following order of the genes typed in the cross and the ratio of recombinants to N2 mice was obtained: centromere—D1Mit34-7/78—D1Mit35-8/90—Nit1-11/91-D1Mit209-telomere. The genetic distances given in centiMorgans (±S.E.) are as follows: centromere—D1Mit209-9.0±3.2—D1Mit35-8.9±3.0—Nit1-12.1±3.4—D1Mit209-telomere. This region of mouse chromosome 1 (1q21-1q23) is syntenic to human chromosome 1q and is consistent with the localization of the human ortholog of Nit1.

Expression and Alternative Splicing of Human and Murine Nit1 Genes

For the human gene, Northern analysis revealed two major transcripts of ˜1.4 kb and ˜2.4 kb in all adult tissues and tumor cell lines tested. A third band of 1.2 kb was observed in adult muscle and heart (FIG. 2B). The longest cDNA (˜1.4 kb) corresponds to the ˜1.4-kb transcript observed on Northern blots. The 1.2-kb band corresponds to transcript 1 on FIG. 3B (see below). It is not known if the ˜2.4-kb RNA represents an additional transcript or an incompletely processed mRNA. No significant variation in human NIT1 mRNA levels was observed in different tissues (FIG. 2B). On the contrary, different mouse tissues showed different levels of expression of Nit1 mRNA (FIG. 2A). The highest levels of Nit1 mRNA were observed in mouse liver and kidney (FIG. 2A, Middle, lanes 5 and 7). Interestingly, the pattern of Nit1 expression was almost identical to the pattern of the expression of Fhit (FIG. 2A, Top and Middle), supporting the hypothesis that the proteins may act in concert or participate in the same pathway.

Analysis of mouse Nit1 ESTs revealed that some transcripts lack exon 2 and encode a 323 amino acid protein. An alternative transcript containing exon 2 encodes a shorter, 290 amino acid protein starting with the methionine 34 (FIG. 1).

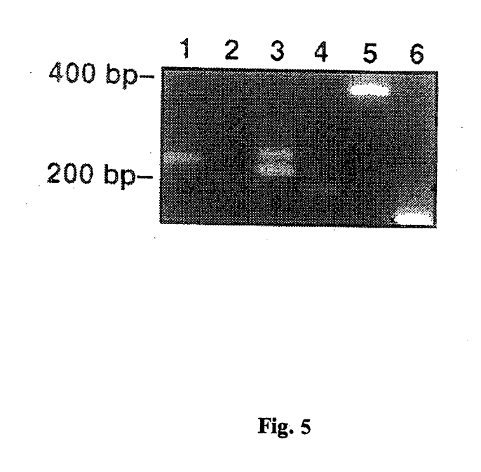

Analysis of human ESTs and 5′ RACE products from HeLa and testis also suggested alternative processing. To investigate this, a series of RT-PCR experiments was carried out. FIG. 5 shows the results obtained from HeLa RNA (similar results were obtained using RNAs from the MDA-MB-436 breast cancer cell line and adult liver). The alternatively spliced transcripts are shown on FIG. 3B. Transcript 1, lacking exon 2, was represented by several ESTs in the Genbank EST database. This transcript probably corresponds to the ˜1.2-kb transcript 15 observed on Northern blots in adult muscle and heart. Transcript 2 encoding the 327 amino acid Nit1 protein (FIG. 1) is a major transcript of human NIT1 at least in the cell lines tested. This transcript lacks exons 1a and 1b. Transcript 3 has exon 1a and 1b; transcript 4 has exon 1a but lacks exon 1b (FIG. 3B). It is not known if transcript 5 (lacking exon 2) starts from exon 1 or 1c.

The alternative initiating methionines of different transcripts are shown on FIG. 3B. Data suggest that at least in COS-7 cells transfected with a construct containing transcript 2, the methionine in exon 3 (shown in transcripts 1 and 3, FIG. 3B) initiates more efficiently than the methionine in exon 2 (FIG. 3B, transcript 2).

Discussion

Although the frequent loss of Fhit expression in several common human cancers is well documented, and results supporting its tumor suppressor activity have been reported, the role of Fhit in normal and tumor cell biology and its mechanism of its action in vivo are unknown. The Ap₃A hydrolytic activity of Fhit seems not to be required for its tumor suppressor function, and it has been suggested that the enzyme-subtract complex is the active form of Fhit. To facilitate an investigation of Fhit function, a model organisms approach was initiated by cloning and characterization of D. melanogaster and C. elegans Fhit genes.

Surprisingly, in flies and worms, Fhit is expressed as a fusion protein with the Fhit domain fused into a “Nit” domain showing homology to plant and bacterial nitrilases. Human and murine NIT1 genes were further isolated. Nit and Fhit are expressed as separate proteins in mammals but, at the mRNA level, are coordinately expressed in mouse tissues.

In several eukaryotic biosynthetic pathways multiple steps are catalyzed by multifunctional proteins containing two or more enzymatic domains. The same steps in prokaryotes frequently are carried out by monoenzymatic proteins that are homologs of each domain of the corresponding eukaryotic protein. For example, Gars, Gart and Airs are domains of the same protein in D. melanogaster and mammals. These domains catalyze different steps in de nova synthesis of purines. In yeast, Gart homolog (Ade8) is a separate protein and Gars and Airs homologs (Ade5 and Ade7) are domains of a bienzymatic protein; in bacteria, all three homologs (PurM, PurN and PurD) are separate proteins. De novo pyrimidine biosynthesis illustrates a similar case. Recently, a fusion protein of a lipoxygenase and catalase, both participating in the metabolism of fatty acids, has been identified in corals. In all of these examples, if domains of a multienzymatic protein in some organisms are expressed as individual proteins in other organisms, the individual proteins participate in the same pathways. This observation and the fact that Fhit and Nit1 exhibit almost identical expression patterns in murine tissues suggest that Fhit and Nit1 participate in the same cellular pathway in mammalian cells.

1. A purified NIT1 gene.

2. The gene of claim 1 which is a human gene.

3. The gene of claim 1 which is a mammalian gene.

4. A purified Nit1 protein.

5. The protein of claim 4 which is a human protein.

6. A purified protein encoded by a nucleic acid having a nucleotide sequence consisting of the coding region of SEQ ID NO:1.

7. An antibody which is capable of binding a Nit1 protein.

8. The antibody of claim 7 which is monoclonal.

9. A molecule comprising a fragment of the antibody of claim 7, which fragment is capable of binding a Nit1 protein.

10. An isolated nucleic acid of less than 100 kb, comprising a nucleotide sequence encoding a Nit1 protein.

11. The nucleic acid of claim 10 in which the Nit1 protein is a human Nit1 protein.

12. A pharmaceutical composition comprising a therapeutically effective amount of a Nit1 protein; and a therapeutically acceptable carrier.

13. A method of treating or preventing a disease or disorder in a subject comprising administering to said subject a therapeutically effective amount of a molecule that inhibits Nit1 function.

14. A method of treating or preventing a disease or disorder in a subject comprising administering to said subject a therapeutically effective amount of a molecule that enhances Nit1 function.

15. A method of diagnosing or screening for the presence of or a predisposition for developing a disease or disorder in a subject comprising detecting one or more mutations in NIT1 DNA, RNA or Nit1 protein derived from the subject in which the presence of said one or more mutations indicates the presence of the disease or disorder or a predisposition for developing the disease or disorder.

16. A method of treating or preventing a disease or disorder in a subject by using a vector containing the NIT1 gene coding sequence.
